SEQUENCE LISTING (1 sequence)

SEQ ID NO: 1 - protein, 141 aa, Artificial sequence
  affibody Z-tri protein
MRGSHEHEHEAENKFNKELIEAAAEIDALPNLNRRQWNAFIKSLVDDPSQSANLLAEAKKLNDAQAPKGGGGSGGGGSGGGGSGSRNLVTAFSNMDDMLQKAHLVIEGTFIYLRDSTEFFIRVRDGWKKLQLGELIPIPAC

(19) United States
(12) Patent Application Publication
(10) Pub. No.: US 2024/0075172 A1
(43) Pub. Date: Mar. 7, 2024

(54) GALLIUM 68 LABELED AFFIBODY PROTEIN PET IMAGING AGENT AND USE THEREOF

(71) Applicant: WEST CHINA HOSPITAL OF SICHUAN UNIVERSITY, Chengdu (CN)

(72) Inventors: Huawei CAI, Chengdu (CN); Xiaofeng LU, Chengdu (CN); Hao YANG, Chengdu (CN); Lin LI, Chengdu (CN); Rong TIAN, Chengdu (CN); Jingqiu CHENG, Chengdu (CN)

(21) Appl. No.: 18/263,453

(22) PCT Filed: Dec. 7, 2021
(86) PCT No.: PCT/CN2021/104028

§ 371 Date: Jul. 28, 2023

(30) Foreign Application Priority Data
Jan. 29, 2021 (CN) 202110126305.7

(51) Int. Cl. A61K 51/10; C07K 16/28

(52) U.S. Cl. CPC A61K 51/1093; C07K 16/2863; C07K 2317/31

(57) ABSTRACT

Disclosed are a gallium 68 labeled affibody protein PET imaging agent and a use thereof; the imaging agent comprises ⁶⁸Ga-Z-tri; the ⁶⁸Ga-Z-tri is obtained by labeling an affibody protein with gallium 68; the affibody protein comprises a PDGFR-β targeting trimer affibody protein Z-tri; and the amino acid sequence listing of the affibody protein Z-tri is as shown in SEQ ID NO. 1. According to the present invention, the utilized PDGFR-β targeting trimer affibody having a unique amino acid sequence has the characteristics of high affinity and high stability compared with a monomer affibody and a dimer affibody, can greatly increase a nuclide labeling rate, and achieves the effectiveness thereof as a PET imaging probe.

### TECHNICAL FIELD

The present invention relates to the field of PET imaging agents for nuclear medicine, and in particular, to a gallium 68 labeled affibody protein PET imaging agent and a use thereof.

### BACKGROUND

Positron emission tomography (PET) is a class of imaging technique for performing non-invasive diagnosis in vivo by using radioactive positron nuclide drugs, such as fluorine 18 (¹⁸F), gallium 68 (⁶⁸Ga) and copper 64 (⁶⁴Cu). A PET probe, built by the positron nuclide in cooperation with different targeting molecule imaging agents, can complete specific function imaging for different diseases and organs, and is of great value in the diagnosis of malignant tumors, neurological and psychotic disorders, and cardiovascular and cerebrovascular diseases.

As an important neovascularization growth factor receptor, platelet derived growth factor receptor-beta (PDGFR-β) has the characteristic of significant high expression in diseases with vessel dysplasia, such as tumors and organic fibrosis. The molecule using the targeting PDGFR-β may be developed for the diagnosis and treatment of the above diseases.

A targeting part of an existing biomolecule PET probe usually adopts a polypeptide or a monoclonal antibody. Although the polypeptide is metabolized quickly in vivo, the affinity and stability in vivo are poor. The monoclonal antibody has better affinity, but due to long cycling time in vivo, it usually takes 24 h or more to be effectively eliminated from the body, and is not conductive to the clinical imaging application.

Affibody molecule (affibody for short) is a class of n-munoglobuln afnity proteins, and a molecular weight of a core targeting binding domain thereof is about 6.5 kD; and the affibody has the specificity (up to the level of nmol/L or pmol/L) equivalent to the antibody, and when applied in the field of radiopharmaceuticals, the affibody has the following unique advantages: \textcircled{1} with a simple structure and a good biological stability, it may be prepared in a large scale by an Escherichia coli prokaryotic expression system; \textcircled{2} it has a low molecular weight, stronger tissue penetration, a quicker speed to eliminate plasma, and low nonspecific binding; and \textcircled{3} it is easily modified, and has better thermal stability and chemical stability compared with a complete antibody, so it is suitable for labeling a plurality of radionuclide. Therefore, PDGFR-β targeting affibody labeled by the positron nuclide ⁶⁸Ga may be developed to become novel radioactive diagnosis drugs, which are used for the diagnosis of tumors, hepatic fibrosis, pulmonary fibrosis and other diseases.

Meanwhile, the monomer affibody molecule and the dimer affibody molecule are used in the existing ⁶⁸Ga-PDGFR-β targeting affibody report, and an affinity constant of the monomer affibody and PDGFR-β is about 249.2 nM, However, although the dimer affibody has a better affinity constant (32.6 nM), this structure is not stable enough in vivo because the dimer structure of the dimer affibody depends on a disulfide bond between the two monomers.

### SUMMARY

In order to solve the above-mentioned problem existing in the prior art, the present invention provides a gallium 68 labeled affibody protein PET imaging agent and a use thereof, the utilized PDGFR-β targeting trimer affibody having a unique amino acid sequence has the characteristics of high affinity and high stability compared with a monomer affibody and a dimer affibody, can greatly increase a nuclide labeling rate, and achieves the effectiveness thereof as a PET imaging probe.

The present invention is achieved by the following technical solution:

An affibody protein is a PDGFR-β targeting trimer affibody protein Z-tri; and the amino acid sequence listing of the affibody Z-tri protein is an amino acid sequence as shown in SEQ ID NO. 1.

A gallium 68 labeled affibody protein PET imaging agent includes ⁶⁸Ga-Z-tri, and the ⁶⁸Ga-Z-tri is obtained by labeling a Z-tri protein with gallium 68.

A preparation method for the gallium 68 labeled affibody protein PET imaging agent, and the preparation method for ⁶⁸Ga-Z-tri includes the following steps of: 1) taking a Z-tri protein to be dissolved in a sodium bicarbonate solution, and adding a tetraazacyclododecane-tetraacetic-acid (DOTA) bifunctional chelating agent for coupling; 2) using a PD-10 desalting column to remove uncombined DOTAs, and obtaining the successfully coupled DOTA-Z-tri protein; and 3) taking the DOTA-Z-tri protein to be dissolved in a natriumaceticum solution, and slowly adding ⁶⁸GaCl₃leacheate to the protein solution according to a ratio for reaction, so as to obtain ⁶⁸Ga-Z-tri.

In step 1), the used DOTA bifunctional chelating agent includes but is not limited to DOTA-N-hydroxysuccinimide (DOTA-NHS), DOTA-N-bromosuccinimide (DOTA-NBS), DOTA-N-chlorosuccinimide (DOTA-NCS), etc.

In step 2), the method for removing the uncombined DOTA chelating agent includes but is not limited to the desalting column, a liquid chromatogram molecular sieve, dialysis and other conventional protein purification methods.

In step 3), a reaction system of the DOTA-Z-tri protein solution and the ⁶⁸GaCl₃ solution is pH 3.5-4.0, 37° C., and a reaction time of 15-30 min.

A use of the gallium 68 labeled affibody protein PET imaging agent, where the imaging agent may be used for the PET imaging of PDGFR-β and highly expressed solid tumors, including but being not limited to spongioblastoma, a colon cancer, osteosarcoma, lung cancer, a liver cancer, a cervical cancer and a breast cancer.

A use of the gallium 68 labeled affibody protein PET imaging agent, where the imaging agent may be used for the PET imaging of PDGFR-β and highly expressed tissue organ fibrotic diseases, including but being not limited to hepatic fibrosis and pulmonary fibrosis.

The present invention has the following beneficial effects:
1. The affibody protein in the present invention is the PDGFR-β targeting trimer affibody protein with the unique amino acid sequence, and not only has better PDGFR-β targeting affinity and stability, but also is suitable for labeling the positron radionuclide.
2. The gallium 68 labeled affibody protein PET imaging agent in the present invention can be used for the PET imaging of the tumors and tissue organ fibrotic diseases in a manner that the PDGFR-β targeting trimer affibody protein with the unique amino acid sequence is labeled with gallium 68.

### BRIEF DESCRIPTION OF DRAWINGS

The drawings illustrated herein are used for providing a further understanding of examples of the present invention, and constitute a part of this application, but do not constitute limitations to the examples of the present invention. In the drawings:

FIG. 1 shows a schematic diagram of an affibody probe in the present invention.

FIG. 2 shows a comparison of a binding force constant between a monomer and PDGFR-beta, between a dimer and PDGFR-beta, and between a trimer and PDGFR-beta in the present invention.

FIG. 3 shows a schematic diagram of a radioactive labeling flow of ⁶⁸Ga-Z-tri.

FIG. 4 shows a labeling efficiency for chromatographic detection of ⁶⁸Ga-Z-tri through a radioactive thin layer.

FIG. 5 shows a PET imaging effect of an imaging agent to mouse with different tumors in the present invention.

FIG. 6 shows the imaging agent in the present invention compared with commonly used clinical PET tumor imaging agent ¹⁸F-FDG used for PET/CT imaging of primary liver cancer lesion of rhesus monkeys.

FIG. 7 shows liver PET imaging that an imaging agent in the present invention is used for carbon tetrachloride induced mouse with hepatic fibrosis.

### DESCRIPTION OF EMBODIMENTS

In order to enable the purpose, the technical solution and the advantage of the present invention to be more clear, the present invention is further described in detail below in combination with the examples and the drawings. The exemplary examples of the present invention and the description thereof are used for explaining the present invention, but do not constitute improper limitations to the present invention.

### Example 1

### Preparation for PDGFR-β Targeting Trimer Affibody Protein (Z-Tri)

### 1.1 Sequence of the Affibody Z-Tri

The amino acid sequence of the affibody protein Z-tri was as shown in SEQ ID NO. 1.

### 1.2 Preparation for Affibody Z-Tri Gene Expression Plasmid

The expression plasmid pQE30-Z-tri of the affibody Z-tri was early designed and built in a laboratory, and saved in a TOP10 strain (TOP10-pQE30-Z-tri). A TOP10-pQE30-Z-tri bacteria solution was taken and inoculated in an LB fluid medium including ampicillin (Amp, 10 μg/ml), to be vibrated and cultured for overnight at 37° C. and 220 rpm until the bacteria solution A₆₀₀ was 1. A part of bacteria solution was taken to extract the pQE30-Z-tri plasmid according to an operation method for a plasmid extraction kit instruction, agarose gel electrophoresis test was performed to extract the plasmid size, the plasmid was delivered to a company for sequencing to verify whether it was pQE30-Z-tri, and the verified pQE30-Z-tri plasmid was saved at 20° C.

### 1.3 Construction of an Expression Strain M15-pQE30-Z-Tri

The pQE30-Z-tri plasmid of which the sequence verification was correct was converted to an Escherichia coli M15 competent cell, and the specific operation steps were as follows:
a) the Escherichia coli M15 competent cell was placed on ice for melting, and 90 μl of molten Escherichia coli M15 competent cell was taken for standby;
b) 10 μl of pQE30-Z-tri was taken to be added into the Escherichia coli M15 competent cell, the mixture was slightly shaken, placed on the ice and in an ice bath for 20 min;
c) the competent cell and a plasmid mixed liquor were placed in a water bath kettle to be in the ice bath for 2 min again after thermally shocked for 45 s, 900 μl of SOC medium was added to be in shake culture for 1 h on a shaking table at 37° C. and 100 rpm;
d) the bacteria solution was centrifuged in conditions of 4,000 g and 5 min, an upper-layer medium was abandoned, and 100 μl of remaining bacteria solution was resuspended;
e) an LB solid medium plate including the ampicillin (Amp, 100 μl/ml) and kanamycin (Kan, 30 μg/ml) was prepared, the resuspended bacteria solution was uniformly smeared on the plate in a bacteria superclean bench for overnight culture at 37° C. in an incubator;
f) five monoclonal colonies were selected to be cultured for overnight respectively at 37° C. and 220 rpm in 5 ml of LB medium (Amp+Kan); and
g) a target fragment was identified whether to be successfully imported and delivered to the company for sequencing through enzyme digestion, 10% of sterilized glycerinum was added into the bacteria solution, subpackaged into 1 ml, and saved at −80° C.

### 2. Expression and Purification of Z-Tri

### 2.1 Induced Expression of Z-Tri Protein

M15-pQE30-Z-tri bacteria solution was selected and cultured for overnight at 37° C. and 220 rpm in 10 ml of LB fluid medium (Amp+Kan), and 1.5 L of medium (Amp+Kan) was added at 37° C. and 220 rpm according to a ratio of 1:300 to enlarge cultivation. When OD600 was about 1, an inducer of isopropyl β-D-thiogalactopyranoside (IPTG) was added into the medium until the concentration was 0.1 mM, and then it was shaken for overnight on the shaking table at 25° C. and 120 rpm.

### 2.2 Purification of Z-Tri Protein

7,000 g of Z-tri protein was centrifuged for 10 min to collect the bacteria in the overnight cultured bacteria solution, the bacteria was resuspended according to lysate (50 mM of phosphate buffer PB, pH8.0, 300 mM of NaCl, and 5 mM of imidazole) of 5 ml/g of bacteria, a protease inhibitor of phenylmethylsulfonyl fluoride (PMSF) was added to 1 mM, and the bacteria was destroyed at a high pressure under conditions of 4° C. and 70 MPa for 4 cycles. Bacteria destroying liquid was used to collect supernate centrifugally under conditions of 4° C., 25,000 g and 15 min for 4 cycles. The bacteria destroying supernate was uniformly mixed with a Ni-NTA resin gel according to a ratio of 50:1, the pH of the mixed solution was adjusted to 8.4 by NaHCO₃, and then the mixture was shaken for overnight on the shaking table at 4° C. and 35 rpm. After the gel supernate mixed solution was collected with a chromatographic column, 40 times of gel column was quickly washed by a scrubbing solution (50 mM of PB pH 8.0, 300 mM of NaCl, and 5 mM of imidazole), and then the gel was slowly washed by eluent (50 mM of PB, pH 8.0, 300 mM of NaCl, and 300 mM of imidazole) for collecting protein until the color reaction of the eluent and the Coomassie brilliant blue G250 was not obvious. The protein was loaded in a dialysis bag with a molecular cut-off of 3.5 K to be dialyzed for overnight by using a phosphate buffer (137 mM of NaCl, 10 mM of Na₂HPO₄, 2.68 mM of KCl, 2 mM of KH₂PO₄, pH 7.4). The dialyzed protein was filtered using 0.22 μm of filtering unit for sterilization, a protein concentration was measured using the Bradford method, and then the protein was subpackaged at −80° C. for storage.

### 2.3 Identification of Z-Tri Protein

Sodium dodecyl sulfate-Polyacrylamide gel electrophoresis (SDS-PAGE) with a spacer gel concentration of 4% and a separation gel concentration of 16% was used as electrophoresis to detect the protein molecular weight size, with a loading amount of 6 μg, 30 min of constant pressure at 80 V, and 30 min of constant pressure at 100 V. The gel was dyed with a commassie blue staining solution (0.25% of R250, 25% of ethyl alcohol and 8% of glacial acetic acid), and it was in a boiling water bath for 15 min. The gel was destained by the destaining solution (25% of ethyl alcohol and 8% of glacial acetic acid) in the boiling water bath, and the gel can be destained repeatedly until the background color was faded.

A polymerization form of the protein was analyzed using an AKTA Pure high performance liquid chromatograph molecular sieve, a loading buffer was the phosphate buffer (15 mM of Na₂HPO₄, 4.02 mM of KCl, 3 mM of KH₂PO₄, 206 mM of NaCl, pH 7.4), and a gel filtration column was Superdex G-75 10/300GL (GE Healthcare), with a loading amount of 0.2 mg and a volume of 100 μl.

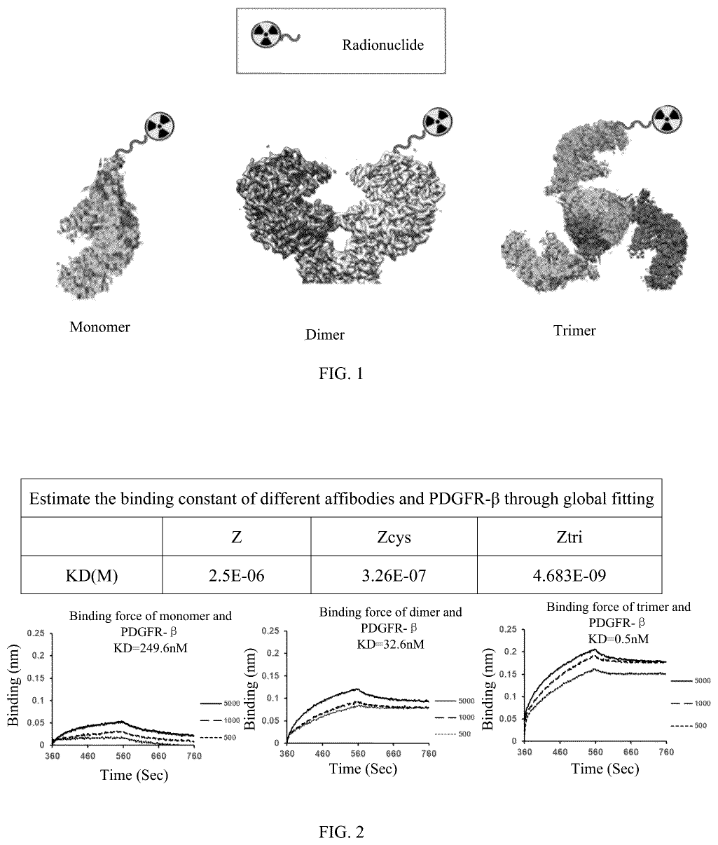

The structural difference among the structural schematic diagram of the trimer affibody protein prepared in the present invention, the monomer (Tolmachev, et al, J Nucl Med 2014; 55:294-300) and the dimer (Cai, et al, Mol. Pharmaceutics 2019, 16, 1950-1957) reported in the existing literature is as shown in FIG. 1. The monomer and the dimer prepared with reference to the method disclosed in the literature are compared with the trimer for the combining capacity to the PDGFR-β, and the comparison results are as shown in FIG. 2. In the present invention, the trimer affibody has the lowest binding constant concentration, indicating the maximum binding force and highest stability thereof.

### Example 2

### Preparation for ⁶⁸Ga-Z-Tri

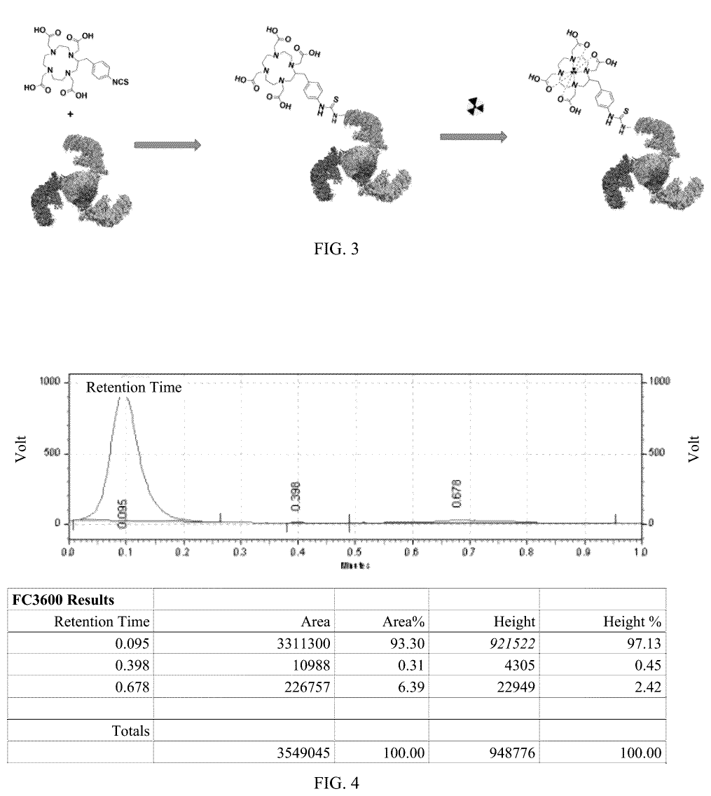

FIG. 3 shows a schematic diagram of a ⁶⁸Ga-Z-tri affibody probe, where the rightmost side shows a trimer affibody probe ⁶⁸Ga-Z-tri in the present invention, and the preparation method thereof is as follows:

### 3.1 Coupling of Z-Tri and a Bifunctional Chelating Agent

After being dissolved in 50 mM of sodium bicarbonate solution (pH 8.2-8.5), and mixed with the bifunctional chelating agent of 20 μL (5 molL) of 1,4,7,10-Tetraazacyclododecane-1,4,7,10-tetraacetic acid, 2-[(4-isothiocyanatophenyl)methyl]-(p-SCN-Bn-DOTA, DOTA for short, the same below), 0.5 mg of Z-tri protein was incubated for 2 hours at 37° C. After the uncombined DOTA was removed using the PD-10 desalting column of the GE company and taking 0.5 M of sodium acetate solution (pH 8.8-9.0) as a flowing phase, the successfully coupled DOTA-Z-tri protein was saved in 0.5 M of sodium acetate solution (pH 8.8-9.0) for standby.

### 3.2 ⁶⁸Ga Labeling of Z-Tri

⁶⁸GaCl₃ eluent was obtained using a 0.1 M of ultra-pure hydrochloric acid leaching Ge—Ga generator, 100 μg of DOTA-Z-tri protein was dissolved in 0.25 mL of sodium acetate solution (0.5 M, pH 8.8-9.0), and according to a volume ratio of the protein to ⁶⁸GaCl₃ as 1:4 (v:v), the ⁶⁸GaCl₃ eluent was slowly added to the protein solution, reacted for 30 min at 37° C. after the pH was determined as 3.5-4.0, and diluted with 0.9% of normal saline for standby. 1-2 μL of reaction product was taken to be sampled on a silica gel plate for radio thin layer chromatography (radio-TLC), a system of 1% ammonium acetate:methanol=50:50 was used, a reference value of the product ⁶⁸Ga-Z-tri Rf was 0-0.2, a reference value of the unlabeled free ⁶⁸Ga ion Rf was 0.7-0.9, and as shown in FIG. 4, it can be seen from the result in the figure that the labeling efficiency of ⁶⁸Ga-Z-tri in the present invention can reach 90% or more.

### Example 3

### PET Imaging Effectiveness Test of ⁶⁸Ga-Z-Tri

1) The ⁶⁸Ga-Z-tri affibody protein was used for performing the PET imaging of a subcutaneous tumor-bearing nude mouse model so as to verify its effectiveness.

Human-derived tumor cell lines were subcutaneously inoculated in respective to build the subcutaneous tumor-bearing nude mouse model, and the subcutaneous tumor-bearing nude mouse models for six malignant tumors, including the malignant gliomas U-87 MG, the colon cancer LS-174T, the lung cancer A549, the osteosarcoma Saos2, the breast cancer MDA-MB-231 and the liver cancer Huh7 were tested.

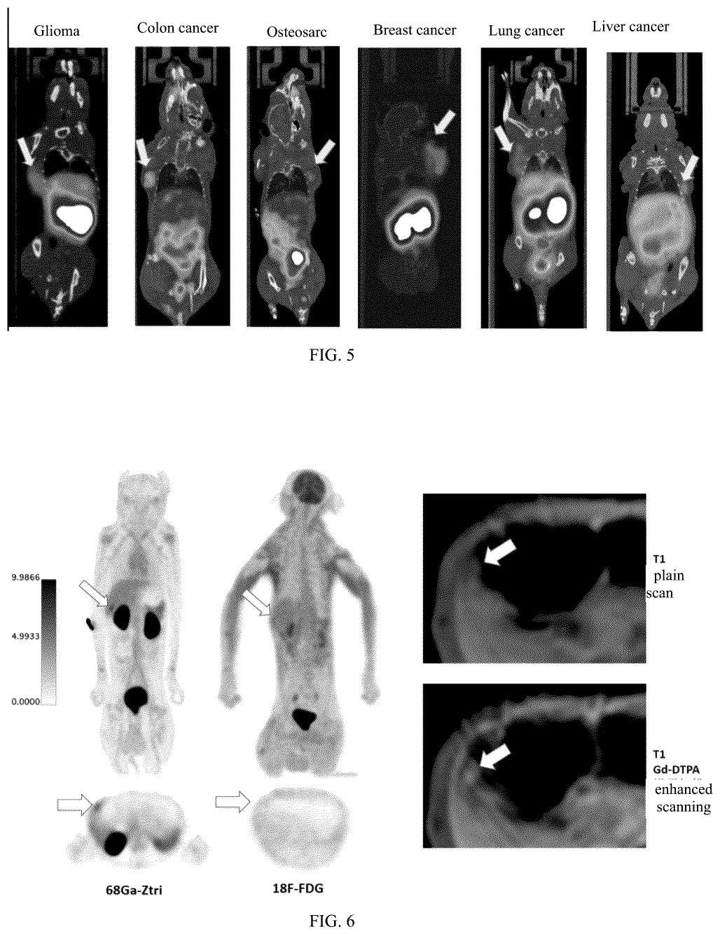

PET imaging was performed after the tumor diameter reached 5-10 mm. The specific steps were as follows: 3.7 MBq of ⁶⁸Ga-Z-tri affibody protein solution (about 1.5 μg of protein amount) was injected in vivo of the tumor-forming nude mouse through caudal vein, and after 1 hour, micro PET-CT was used for imaging. As shown in FIG. 5, where the arrow points is a tumor, it can be clearly seen from the figure that most drugs are metabolized in vivo through a kidney-bladder pathway, while the corresponding subcutaneous tumor sites of the mouse all occur obvious radioactive uptake, proving that the ⁶⁸Ga-Z-tri affibody protein may be used for PET imaging of the tumors.
2) The ⁶⁸Ga-Z-tri affibody protein was used for performing the PET imaging for the primary liver cancer of a rhesus monkey so as to verify its effectiveness.

The rhesus monkey was intravenously injected with about 20 μg of ⁶⁸Ga-Z-tri affibody protein with reference to the human imaging dose (3.7 MBq/Kg body weight), and after 1 hour, PET-CT whole body imaging was performed. As shown in FIG. 6, a primary liver cancer lesion with a diameter of about 6.5 mm of the right lobe of the liver can be clearly seen at the arrow, and by drawing area-of-interest (ROI), it may find that a multiple ratio of a maximum value (SUVmax) of a drug uptake standard value in the ROI of the lesion site to a corresponding value of the surrounding normal liver area is 2.77 times, with a better PET imaging effect. Compared with the tumor imaging agent 18F-FDG commonly used in clinic, ⁶⁸Ga-Z-tri imaging result shows a better imaging effect on the liver cancer lesion. Magnetic resonance (MR) imaging result has verified that this lesion is Ti enhanced malignant lesion. The excretion pathway of the excess drugs is consistent with that of the mouse, and they are excreted by kidney. In addition, due to a greater body, the tumor lesion imaging effect of the liver site of the animal is far superior to that of the mouse model with the increase of the distance from the kidney position to the tumor position.
3) The ⁶⁸Ga-Z-tri affibody protein was used for performing the PET imaging for the mouse hepatic fibrosis so as to verify its effectiveness.

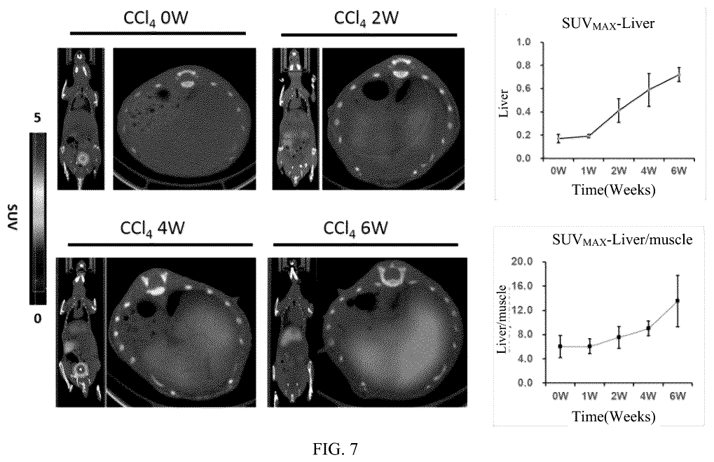

The general carbon tetrachloride CCl4 modeling method was referred for the induced establishment of the mouse hepatic fibrosis model. C57 strain mouse were selected, either gender, to be injected with 20% of carbon tetrachloride olive oil solution once three days through enterocoelia according to 5 mL/kg of weight, continuous administration lasted for six weeks, and 3.7 MBq of ⁶⁸GaZtri affibody protein solution (about 1.5 μg of protein amount) was respectively injected through caudal vein before administration, 2, 4, 6 week after administration for PET imaging. The imaging result is as shown in FIG. 7, it can be seen that the maximum value (SUV max) of the drug uptake standard value to the ⁶⁸Ga-Z-tri imaging agent in the liver region of interest and a ratio of the liver to the muscle appear a corresponding upward trend with the aggravated induction degree of the hepatic fibrosis. It has verified that ⁶⁸Ga-Z-tri affibody protein can be used for PET imaging of the hepatic fibrosis.

FIG. 5 and FIG. 7 themselves are colorful pictures, and the applicant can only submit the processed black and white pictures due to the related provisions of the Patent Laws. If these pictures are regarded to be unclear or cannot display the above-mentioned effects, the applicant may submit the colorful pictures for supplementary statement.

The purpose, the technical solution and the beneficial effects of the present invention are further described in detail through the specific implementation modes above. It should be understood that the above is only alternative embodiment of the present invention and not intended to limit the protective scope of the present invention. Any modifications, equivalent replacements, improvements and the like made within the spirit and principle of the present invention shall fall within the protective scope of the present invention.

We claim:

1. An affibody protein, comprising a PDGFR-β targeting trimer affibody protein Z-tri; wherein the amino acid sequence of the affibody Z-tri protein is an amino acid sequence as shown in SEQ ID NO. 1.

2. A gallium 68 labeled affibody protein PET imaging agent, comprising ⁶⁸Ga-Z-tri, wherein the ⁶⁸Ga-Z-tri is obtained by labeling the affibody protein of claim 1 with gallium 68.

3. A preparation method for the gallium 68 labeled affibody protein PET imaging agent of claim 2, wherein the preparation method for ⁶⁸Ga-Z-tri comprises the following steps of: 1) taking a Z-tri protein to be dissolved in a sodium bicarbonate solution, and adding a bifunctional chelating agent tetraazacyclododecane-tetraacetic-acid (DOTA), and incubating after mixing; 2) removing uncombined chelating agent, obtaining the successfully; and 3) taking the DOTA-Z-tri protein to be dissolved in a natriumaceticum solution, and slowly adding ⁶⁸GaCl₃leacheate to the protein solution according to a ratio, adjusting a pH value to 3.5-4.0, and reacting at 37° C. so as to obtain ⁶⁸Ga-Z-tri.

4. A use of the gallium 68 labeled affibody protein PET imaging agent of claim 2, wherein the imaging agent is used for the PET imaging of solid tumors with high PDGFR-β expression, including spongioblastoma, a colon cancer, a lung cancer, a liver cancer, a cervical cancer, or a breast cancer.

5. A use of the gallium 68 labeled affibody protein PET imaging agent of claim 2, wherein the imaging agent is used for the PET imaging of tissue organ fibrotic diseases with high PDGFR-β expression, including hepatic fibrosis or pulmonary fibrosis.
